(19) United States
(12) Patent Application Publication
(10) Pub. No.: US 2007/0212415 A1
(43) Pub. Date: Sep. 13, 2007

(54) SUSTAINED-RELEASE TABLET CONTAINING DOXAZOSIN MESYLATE

(75) Inventors: Jun Sang Park, Seongnam-si (KR); Ji-Yeon Shim, Seongnam-si (KR); Hun Sik Wang, Seongnam-si (KR); Min Chang Kwon, Seoul (KR)

Correspondence Address: SUGHRUE MION, PLLC, 2100 PENNSYLVANIA AVENUE, N.W., SUITE 800, WASHINGTON, DC, 20037, US

(73) Assignee: GL PharmTech Corp., Seongnam-si (KR)

(21) Appl. No.: 11/575,122

(22) PCT Filed: Aug. 26, 2005
(86) PCT No.: PCT/KR05/02841

§ 371 Date: Mar. 12, 2007

(30) Foreign Application Priority Data
Sep. 15, 2004 (KR) 10-2004-0073976

(51) Int. Cl. A61K 31/517; A61K 9/22

(52) U.S. Cl. 424/468; 514/252.17

(57) ABSTRACT

The present invention relates to a sustained-release tablet containing doxazosin mesylate, and more particularly to a sustained-release tablet of which drug release rate maintains constant for 8 hours and longer.

### TECHNICAL FIELD

The present invention relates to a sustained-release tablet containing doxazosin mesylate, and more particularly to a sustained-release tablet showing a constant release rate of drug for more than 8 hours.

### BACKGROUND ART

Sustained-release dosage forms generally control the rate of drug absorption, so as to avoid excessive drug absorption while maintaining effective blood concentration of the drug to provide a patient with a consistent therapeutic effect over the extended time.

Besides reducing the frequency of dosing and providing a more consistent therapeutic effect, sustained-released dosage forms generally help reduce side effects caused by a drug and enhance the patient's compliance.

The chemical of doxazosin is 1-(4-amino-6,7-dimethoxy-2-quinazolinyl)-4-[(2,3-dihydro-1,4-benzodioxin-2-yl)carbonyl]piperazine. It is generally used as a form of pharmaceutically acceptable salts, particularly as a form of doxazosin mesylate in order to improve its solubility in water. Doxazosin is in a class of drugs called alpha-adrenergic receptor antagonists. Doxazosin is used to treat hypertension and benign prostatic hyperplasia. It relaxes the muscles in the prostate and bladder neck, making it easier to urinate.

Anti-hypertensives are used to maintain a constant blood pressure. The U.S. Food and Drug Administration (FDA) recommended that blood concentration immediately prior to next dosing keep minimum effective blood concentration, with the ratio of trough and peak blood concentrations not less than ½ in order that the risks of postural hypotension or ischemic attack and blood pressure fluctuation should be minimized.

Anti-hypertensives should have maintained adequate bioavailability of the drug to achieve a constant therapeutic effect over the extended time. Preferably, anti-hypertensives may have to be administered once a day as a formula to ensure patient's compliance and convenience in dosing, and a therapeutically effective blood concentration of drug should be maintained for at least 24 hours.

Therefore, doxazosin mesylate is preferably prepared as a sustained-release dosage form with the following conditions to be met:

Firstly, doxazosin mesylate should give essentially a zero order release profile to ensure a therapeutically effective blood concentration of drug for a longer period of time, and

Secondly, the rate of drug released should remain constant for at least 8 hours in drug delivery systems having a zero order release profile, thereby maintaining a therapeutically effective blood concentration of drug for at least 24 hours.

In the following equation, a zero order means n=1; if n=1, the rate of drug released remains constant over the entire delivery profile irrespective of passage of time (hence, Y may be calculated until it reaches 60%). If n is in the range of 0.8 to 1.2, the drug is considered to be released at a favorable zero order release profile.
Y=ktⁿ

(Y: amount of drug released (%), k: rate constant, n: release exponent, t: time)

It usually takes 9 to 13 hours for orally administered doxazosin mesylate dosage forms to pass stomach and small intestine into colon. The dosage forms, generally, pass through the colon after 16 hours at the longest, even considering a maximum gastric emptying time of about 5 hours. In this respect it is not desirable that the total release time of the sustained-release drug exceeds 24 hours. Therefore, it is preferable that total releasing time of a sustained-release doxazosin mesylate tablet of the present invention should be set at the range of 8-24 hours.

In order to achieve such objective, a wide variety of sustained-release dosage forms have been developed; a mechanical or osmotic pump, diffusion-controlling membrane, a capsule having a drug-containing core coated with membrane, and a matrix where drug is dispersed within a drug release control layer.

The U.S. Pat. No. 4,765,989 discloses osmotic system(OROS) so as to achieve the zero order delivery but it fails to release the drug for initial 2 hours after its administration. Even after drug release, it could not release about 5-10% of the total drug and excreted from the body. As a result, the assessment for its bioavailability is deemed inefficient. Furthermore, the actual application has limitation in terms of more complicated production process and higher manufacturing cost.

To solve drawbacks of the said patent, various approaches have been directed at developing a sustained-release preparation in a matrix form in order that (1) a constant drug release profile may be ensured theoretically, (2) manufacturing method is easy, and (3) production cost is economically feasible. Hence, pharmaceutical manufacturers have mainly used hydrophilic polymers such as hydroxypropylmethyl cellulose (HPMC).

The Korean Patent Publication No. 1993-46 disclosed the use of a sustained-release pharmaceutical carrier which comprises HPMC, hydroxypropyl cellulose and carboxyvinyl polymer. The U.S. Pat. No. 4,389,393 disclosed the formulation of a sustained-release preparation comprising one or more HPMCs, or a mixture of one or more HPMCs and other celluloses by 30 wt % or more. The said HPMC matrix formulation has been recognized to have some disadvantages in that (1) initial burst of drug occurs in early stage due to the rule of diffusion, and (2) the drug release rate decreases along with the passage of time, whereby the zero order profile may not be followed until the 100% of drug completely releases.

To provide an improved zero order drug delivery system, the U.S. Pat. No. 6,156,343 disclosed the manufacture of a sustained-release tablet having a coating layer, comprising an water-insoluble polymer and at least one selected from the group consisting of a water-soluble polymer and an enteric polymer. The U.S. Pat. No. 6,500,459 disclosed a sustained-release tablet having a multi-coating layer. The U.S. Pat. No. 5,422,123 disclosed a sustained-release tablet having a core and support.

The Korean Patent No. 10-0422418 disclosed a sustained-release film-coated tablet containing dihydropyridine derivative as an active ingredient, wherein it comprises of a matrix and a film-coating layer. The matrix contains HPMC, glyceryl behenate, polyvinyl pyrrolidone, and calcium hydrogen phosphate. In addition, the film-coating layer includes HPMC as a film-forming agent, polyethylene glycol 6000 as a plasticizer, titanium dioxide as an opacifier. The above prior technologies have yet coped with some drawbacks in actual application, such as (1) the failure of drug release for over 8 hours on a zero order release profile, (2) more complicated production process requiring additional coating process, and a higher production cost.

The U.S. Pat. No. 5,393,765 disclosed a sustained-release dosage form using HPMC having viscosity of 50 to 100 cps, wherein the matrix may be erodible to provide a zero order release profile instead of drug diffusion in the matrix. However, it has a limit in application, since the zero order release maintains for below 8 hours. Thus, a therapeutically effective blood concentration of the drug cannot be maintained for 24 hours.

The Korean Patent No. 10-0234446 (the U.S. Pat. No. 5,126,145) disclosed a sustained-release tablet containing HPMC, a binding agent, hydrophobic component, and a water-soluble drug. According to the conventional method of the sustained-release dosage form, a water-soluble drug accounts for 50 to 85%, followed by HPMC with high viscosity in 5 to 30%, HPMC with low viscosity in 2 to 15% as a binding agent, and hydrogenated vegetable oil in 2 to 20% as a hydrophobic component.

Despite the fact that the above prior arts have been designed to give essentially a zero order release profile, there are limitations in applying doxazosin mesylate in that a complete zero order delivery has not been achieved, or the drug was not released for over 8 hours on a zero order release.

In order to overcome the aforementioned shortcomings, the inventors have tried and succeeded in manufacturing a sustained-release doxazosin mesylate tablet, comprising the steps of:

a) forming granules comprising both HPMCs with high and low viscosity (7,500 to 14,000 cps and 40 to 60 cps, respectively), and

b) mixing the above granule with the said HPMC with low viscosity (40 to 60 cps) and glyceryl behenate.

As a result, the sustained-release doxazosin mesylate tablet was released for over 8 hours on a zero order release profile and thus, the present invention was consummated.

### DISCLOSURE OF INVENTION

Technical Problem

The objective of the present invention is to provide a once-daily sustained-release doxazosin mesylate tablet that can display a zero order release profile for over 8 hours and preparation method thereof.

Technical Solution

In order to achieve the above objective, the present invention provides a sustained-release doxazosin mesylate tablet prepared by the steps of comprising:

a) granulating the mixture comprising doxazosin mesylate and both HPMCs having viscosity of 7,500 to 14,000 cps and 40 to 60 cps, respectively, and

b) mixing the above granules with the said HPMC having viscosity of 40 to 60 cps and glyceryl behenate and tabletting thereof.

The present invention also provides a method of manufacturing a sustained-release doxazosin mesylate tablet, comprising the steps of:

a) granulating the mixture comprising doxazosin mesylate and both HPMCs having viscosity of 7,500 to 14,000 cps and 40 to 60 cps, and

b) mixing the above granule with the said HPMC having viscosity of 40 to 60 cps and glyceryl behenate and tabletting thereof.

The inventors have succeeded in manufacturing a sustained-release tablet that can overcome the drawbacks of prior arts and display a zero order release profile for over 8 hours based on the following conditions:

Firstly, the sustained-release tablet should be eroded at a proper rate when administered in the body.

Secondly, the initial burst of matrix comprising hydrophilic polymers should be prevented.

Thirdly, the drug release rate out of granule can be controlled properly and that way, the drug release time at zero order rate should be maintained for 8-24 hours.

The inventors have learnt that the prior arts may not be applicable to achieve a zero order delivery of a sustained-release doxazosin mesylate tablet. Further, it was confirmed that the desired zero order release profile of the present invention may be achieved by adding a certain amount of HPMC having low viscosity of 40 to 60 cps to doxazosin mesylate granules. Furthermore, it was noted that the rate of drug release was not decreased in later stage even in the presence of a large amount of HPMC with high viscosity polymer that can significantly extend the total release time of drug.

The present invention is characterized by the manufacture of granules comprising doxazosin mesylate and both HPMCs with high and low viscosity to ensure a proper drug release rate.

The present invention is also characterized by the manufacture of a sustained-release tablet by adding the said HPMC with low viscosity and glyceryl behenate to the granule so as to control the erosion rate and to prevent initial burst.

Because of the characteristics described above, the present invention provides a sustained-release tablet that can display a zero order release profile for over 8 hours.

The sustained-release tablet of the present invention is described in more detail as set forth hereunder.

The present invention includes granules prepared by mixing and kneading doxazosin mesylate with HPMC having viscosity of 7,500 to 14,000 cps and HPMC having viscosity of 40 to 60 cps.

According to the present invention, the viscosity of HPMC indicates the centipoises of a 2% aqueous solution at 20° C.; the HPMC with low viscosity refers to the HPMC having the viscosity of 40 to 60 cps (trade name: Metolose 60SH50 (Shin-Etsu, Japan)), and the HPMC with high viscosity refers to the HPMC having the viscosity of 7,500 to 14,000 cps (trade name: Metolose 60SH10,000 (Shin-Etsu, Japan) or Methocel E10M Premium CR (Dow Chemical, U.S.A.)).

A tablet prepared only with the said granules may control the drug release to some extent, but the tablet release rate cannot ensure a proper zero order profile for over 8 hours.

Furthermore, the tablet prepared only with the granules to contain HPMC with high viscosity is disadvantageous in that a rapid drug release does not allow doxazosin mesylate to achieve a proper zero order delivery for over 8 hours. By contrast, the tablet prepared only with the granules to contain HPMC with high viscosity without HPMC with low viscosity is also disadvantageous in that a low drug release rate does not allow the total drug amount to be completely released within 24 hours after administration, and therefore the remained drug in the tablet may be excreted. Thus, these tablets are not desirable for once a day.

The HPMC with high viscosity is contained by 3 to 30 w/w % in the above-invented granules, preferably by 5 to 20 w/w %. The HPMC with low viscosity is contained by 15 to 30 w/w % in the granules, preferably by 20 to 25 w/w %.

The said granules comprising doxazosin mesylate are contained by 40 to 80 w/w % in the total tablet weight, preferably by 50 to 60 w/w %.

It is preferable that one or more excipients contained in the granules are selected from the group consisting of lactose and microcrystalline cellulose.

The present invention can extend the desirable release time at zero order rate by additionally mixing the HPMC with low viscosity with the said granules.

The HPMC with low viscosity, which is added in post-mixing process, can extend the drug release time in a proper manner and serve to maintain the drug delivery for over 8 hours on a zero order release profile.

The postly-mixed HPMC with low viscosity is the same as the said HPMC with low viscosity into the said granules. In case of using HPMC with higher viscosity, the excessively extended drug release time does not allow the total amount of drug to be released up to 24 hours. By contrast, in case of using HPMC with lower viscosity, the drug release time is further shortened or a zero order release profile may not be ensured.

The HPMC with low viscosity to be added in post-mixing is contained by 10 to 40 w/w % in total tablet weight, preferably by 20 to 30 w/w %.

In order to prevent the initial burst of drug, the present invention is characterized by the use of glyceryl behenate, a hydrophobic lubricant in the post-mixing process. It may be contained by 5 to 30 w/w % in the total tablet weight.

The present invention also provides a method of manufacturing a sustained-release doxazosin mesylate tablet, comprising the steps of:

a) granulating the mixture comprising doxazosin mesylate and both HPMCs having viscosity of 7,500 to 14,000 cps and 40 to 60 cps, and

b) mixing the above granules with HPMC having viscosity of 40 to 60 cps and glyceryl behenate and tabletting thereof.

According to the present invention, HPMC having viscosity of 7,500 to 14,000 cps is suspended or dissolved in an organic solvent, then is added to the mixture comprising doxazosin mesylate and HPMC with low viscosity. The granules obtained is dried and sieved into suitable size. Alternatively, both doxazosin mesylate and HPMC having viscosity of 7,500 to 14,000 cps are suspended or dissolved together in an organic solvent, then is added to the mixture comprising HPMC with low viscosity.

The granules obtained is dried and sieved into suitable size.

The HPMC with high viscosity having viscosity of 7,500 to 14,000 cps is hard to dissolve in water due to its high viscosity, so it is preferable to be suspended or dissolved in an organic solvent.

According to the present invention, an organic solvent may be selected from a group consisting of ethanol, isopropanol, propanol, chloroform and methylene chloride, either alone or together. Further, any of pharmaceutically acceptable solvents may be used to suspend or dissolve the drug and HPMC, but not limited to the afore-mentioned organic solvents.

The said granules can be prepared by a conventional granulator in pharmaceutical industry. The examples of the granulator used for the present invention include an extrusion granulator such as screw-type extrusion granulator, cylindrical granulator, and oscillating granulator, or other mechanical apparatus such as vertical granulator (trade name: Hi-speed Mixer, Freund, Japan), and a vertical granulator is preferably used.

The said granules are mixed with HPMC having viscosity of 40 to 60 cps and glyceryl behenate, followed by the tabletting process to prepare a sustained-release tablet.

Advantageous Effects

The sustained-release tablet containing doxazosin mesylate according to the present invention can display a zero order release for at least 8 to 24 hours. As the drug release rate is almost independent of the passage of time, a therapeutically effective and consistent blood concentration can be maintained for an extended period of time. As a result, the sustained-release tablet of the present invention is advantageous in that it may provide a patient with a consistent therapeutic effect over the extended duration of effect and it helps reduce side effects caused by drug.

In this context, the present invention can provide a preparation suitable for an once-daily formulation that maintains a therapeutically effective blood concentration of doxazosin mesylate for 24 hours in the body.

### BRIEF DESCRIPTION OF THE DRAWINGS

FIG. 1 is the results of drug release test in Examples 1 to 5; and

FIG. 2 is the results of drug release test in Comparative Examples 1 to 3.

### BEST MODE FOR CARRYING OUT THE INVENTION

The present invention is described in more detail by the following Examples, which should not be considered to limit the scope of the present invention.

### Example 1 & 2

According to the composition (unit: g) of Table 1 below, HPMC with high viscosity (Methocel E10M Pr. CR) was suspended to 80 ml of ethanol; then this was added into the mixture of doxazosin mesylate, lactose, microcrystalline cellulose and HPMC with low viscosity (Metolose 60SH50); and the whole was granulated, dried, and sieved. Then, HPMC with low viscosity (Metolose 60SH50) and glyceryl behenate were added and mixed together; and the whole was tableted into a round tablet.

### Example 3

According to the composition (unit: g) of Table 1 below, doxazosin mesylate and HPMC with high viscosity (Methocel E10M Pr. CR) were suspended to 80 ml of ethanol; then into this mixture, lactose, microcrystalline cellulose and HPMC with low viscosity (Metolose 60SH50) were added; and the whole was granulated, dried, and sieved. Then, HPMC with low viscosity (Metolose 60SH50) and glyceryl behenate were added and mixed together; and the whole was tableted into a round tablet.

### Example 4

According to the composition (unit: g) of Table 1 below, HPMC with high viscosity (Methocel E10M Pr. CR) was suspended to 80 ml of ethanol; then into this mixture, doxazosin mesylate, lactose, microcrystalline cellulose and HPMC with low viscosity (Metolose 60SH50) were added; and the whole was granulated, dried, and sieved.

Then, HPMC with low viscosity (Metolose 60SH50) and glyceryl behenate were added and mixed together; and the whole was tableted into a round tablet.

### Example 5

According to the composition (unit: g) of Table 1 below, 10 g of HPMC with high viscosity (Methocel E10M Pr. CR) was suspended to 80 ml of ethanol, then into this mixture, doxazosin mesylate, lactose, microcrystalline cellulose, HPMC with high (Methocel E10M Pr. CR) and low (Metolose 60SH50) viscosity were added; and the whole was granulated, dried, and sieved. Then, HPMC with low viscosity (Metolose 60SH50) and glyceryl behenate were added and mixed together; and the whole was tableted into a round tablet.

TABLE 1
Composition for Examples 1 to 5 (unit: g)

| | | Example 1 | Example 2 | Example 3 | Example 4 | Example 5 |
|---|---|---|---|---|---|---|
| Granulating | doxazoc in mesylate | 5.09 | 5.09 | 5.09 | 5.09 | 5.09 |
| Step | HPMC with high viscosity | 8.6 | 10 | 18 | 8.6 | 55 |
| | (Methocel E10M Pr. CR) | | | | | |
| | HPMC with low viscosity | 40 | 40 | 40 | 40 | 40 |
| | (Metolose 60SH50) | | | | | |
| | lactose | 72 | 90 | 128 | 72 | 87 |
| | microcrystalline cellulose | 24 | 30 | 50 | 24 | 9 |
| Post-mixing | HPMC with low viscosity | 63 | 100 | 118 | 62.5 | 70 |
| Step | (Metolose 60SH50) | | | | | |
| | glyceryl behenate | 88 | 40 | 23 | 88 | 23 |

### Experimental Example 1

### Dissolution Test

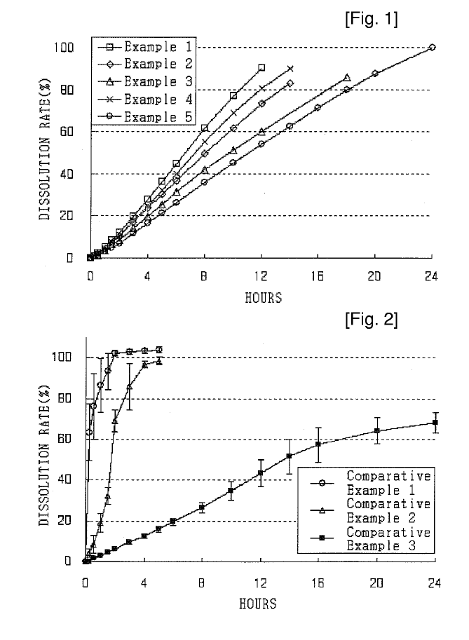

Dissolution tests of Examples 1 to 5 were performed at 50 rpm according to the Korean Pharmacopoeia Dissolution Test Method No. 2 in 900ml of a pH 6.8 phosphate buffer solution (Korean Pharmacopoeia, Dissolution Test Method No. 2 solution). The results are shown in Table 2 and FIG. 1, respectively.

TABLE 2
Dissolution rate per hour (%/hr) and n-value

| Hour | Example 1 | Example 2 | Example 3 | Example 4 | Example 5 |
|---|---|---|---|---|---|
| 0.5 | 4.6 | 4.1 | 2.7 | 4.1 | 2.8 |
| 1 | 5.6 | 4.9 | 4.3 | 5.0 | 3.3 |
| 1.5 | 6.5 | 5.6 | 5.6 | 5.8 | 3.5 |
| 2 | 7.2 | 6.2 | 4.2 | 6.4 | 4.4 |
| 3 | 7.7 | 6.5 | 5.6 | 6.9 | 4.6 |
| 4 | 8.0 | 6.9 | 5.7 | 7.2 | 4.8 |
| 5 | 8.5 | 6.3 | 5.7 | 7.6 | 5.0 |
| 6 | 8.6 | 6.5 | 6.0 | 7.7 | 4.8 |
| 8 | 8.4 | 6.4 | 5.2 | 7.5 | 4.9 |
| 10 | 7.7 | 6.2 | 4.7 | 6.9 | 4.6 |
| 12 | 6.7 | 5.7 | 4.5 | 6.0 | 4.5 |
| n | 1.20 | 1.15 | 1.19 | 1.15 | 1.13 |

According to the test results shown in Table 2 and FIG. 1, those tablets prepared in Examples 1 to 5 are recognized to be eroded at a proper rate; so it is expected that they will be eroded at a proper rate when dosed in the body. In addition, the sustained-release tablet of the present invention could inhibit initial burst and maintained a constant drug release rate for 8 to 12 hours or longer; and it is recognized that the drug is released at zero order rate maintaining n-values in the range of 0.8 and 1.2 for at least 8 hours. Particularly in Example 5 as can be known from FIG. 1, the drug is released at zero order rate for 8 hours or up to 24 hours.

### Comparative Example 1

According to the composition (unit: g) of Table 3 below, a sustained-release tablet was prepared in the same method as Example 1 except for post-mixing of HPMC with low viscosity and glyceryl behenate.

### Comparative Example 2

A sustained-release tablet was prepared with the composition (unit: g) of Table 3 below according to the method of Example 1 except for HPMC with low viscosity in post-mixing.

### Comparative Example 3

A sustained-release tablet was prepared with the composition (unit: g) of Table 3 below according to the method of Example 1 except that HPMC having viscosity of 400 cps (Metolose 60SH400) was used instead of HPMC with low viscosity in post-mixing.

TABLE 3
Composition for comparative examples 1 to 3 (unit: g)

| | | Comparative | Comparative | Comparative |
|---|---|---|---|---|
| | | Example 1 | Example 2 | Example 3 |
| Granulating | doxazocin mesylate | 5.09 | 5.09 | 5.09 |
| Step | HPMC with high viscosity | 10 | 100 | 8.6 |
| | (Methocel E10M Pr. CR) | | | |
| | HPMC with low viscosity | 40 | 40 | 40 |
| | (Metolose 60SH50) | | | |
| | lactose | 87.21 | 87.21 | 87.21 |
| | microcrystalline cellulose | 8.7 | 8.7 | 8.7 |
| Post-mixing | HPMC with low viscosity | — | — | — |
| Step | (Metolose 60SH50) | | | |
| | HPMC with low viscosity | — | — | 70 |
| | (400 cps of viscosity, Metolose | | | |
| | 60SH400) | | | |
| | glyceryl behenate | — | 20 | 20 |

### Experimental Example 2

### Dissolution Test

Dissolution tests were accomplished on above Comparative Examples 1 to 3 each by the same method as Experimental Example 1. Results thereof are illustrated in FIG. 2 and in Table 4.

TABLE 4
Dissolution rate per hour (%/hr) and n-value

| | Comparative | Comparative | Comparative |
|---|---|---|---|
| Hour | Example 1 | Example 2 | Example 3 |
| 0.25 | 15.94 | 253.59 | 0.00 |
| 0.5 | 17.48 | 50.97 | 6.22 |
| 1 | 21.23 | 20.72 | 2.98 |
| 1.5 | 26.27 | 13.65 | 3.18 |
| 2 | 74.25 | 18.02 | 3.21 |
| 3 | 16.71 | 0.57 | 3.12 |
| 4 | 10.79 | 0.43 | 3.24 |
| 5 | 1.83 | 0.50 | 3.35 |
| 6 | — | — | 3.36 |
| 8 | — | — | 3.63 |
| 10 | — | — | 4.10 |
| 12 | — | — | 4.33 |
| n | — | 1.307 | 0.910 |

In cases of Comparative Examples 1 and 2 where the granules containing doxazosin mesylate was used alone or granules mixed only together with glyceryl behenate was used, respectively, drug release ended completely within 6 hours as well as n-value escaped from the range of 0.8 to 1.2. In particular, in case of Comparative Example 1, initial burst was inclined to increase excessively not to calculate n-value itself.

The results of Comparative Example 3 containing HPMC of 400 cps in post-mixing showed that drug release was extended excessively, so more than 30% of total drug could not be released even after 24 hours.

1. A sustained-release tablet prepared by the steps of comprising:
a) obtaining the granules by granulating the mixture comprising doxazosin mesylate and both hydroxypropylmethyl celluloses having viscosity of 7,500 to 14,000 cps and 40 to 60 cps, and
b) mixing the above said granules with hydroxypropylmethyl cellulose having viscosity of 40 to 60 cps and glyceryl behenate; and
wherein the dissolution test results of the sustained-release tablet according to the Korean Pharmacopoeia's Dissolution test method II (50rpm, 900ml of pH 6.8 buffer solution) show that the total drug release time is 8-24 hours, and n-value defined in the following equation is 0.8 to 1.2:
Y=ktⁿ (Y: amount of drug released (%), k: rate constant, n: release exponent, t: time).

2. The sustained-release tablet according to claim 1, wherein the said granules containing doxazosin mesylate include hydroxypropylmethyl cellulose having viscosity of 7,500 to 14,000 cps by 3 to 30 w/w % and hydroxypropylmethyl cellulose having viscosity of 40 to 60 cps by 15 to 30 w/w %.

3. The sustained-release tablet according to claim 1, wherein the said granules containing doxazosin mesylate account for 40 to 80 w/w % in the total tablet weight.

4. The sustained-release tablet according to claim 1, wherein the said granules containing doxazosin mesylate include one or more excipients selected from the group consisting of lactose and microcrystalline cellulose.

5. The sustained-release tablet according to claim 1, wherein the post-mixed hydroxypropylmethyl cellulose having viscosity of 40 to 60 cps in b) is contained by 10 to 40 w/w % in the total tablet weight.

6. A method for preparing the sustained-release tablet, comprising the steps of:
a) obtaining the granules by granulating the mixture comprising doxazosin mesylate and both hydroxypropylmethyl celluloses having viscosity of 7,500 to 14,000 cps and 40 to 60 cps, and
b) mixing the above said granules with hydroxypropylmethyl cellulose having viscosity of 40 to 60 cps and glyceryl behenate and tabletting thereof.

7. The method for preparing sustained-release tablet according to claim 6, wherein hydroxypropylmethyl cellulose having viscosity of 7,500 to 14,000 cps is suspended or dissolved in organic solvent, and then doxazosin mesylate and hydroxypropylmethyl cellulose having viscosity of 40 to 60 cps are mixed and granulated.

8. The method for preparing sustained-release tablet according to claim 6, wherein the hydroxypropylmethyl cellulose having viscosity of 7,500 to 14,000 cps and doxazosin mesylate are suspended or dissolved in organic solvent, and then hydroxypropylmethyl cellulose having viscosity of 40 to 60 cps is mixed and granulated.

9. A sustained-release tablet, comprising the steps of:
a) obtaining the granules by granulating the mixture comprising doxazosin mesylate, both hydroxypropylmethyl celluloses having viscosity of 7,500 to 14,000 cps and 40 to 60 cps and one or more excipients selected from the group consisting of lactose and microcrystalline cellulose, and
b) mixing the above said granules with hydroxypropylmethyl cellulose having viscosity of 40 to 60 cps and glyceryl behenate; and
wherein the dissolution test results of the sustained-release tablet according to the Korean Pharmacopoeia's Dissolution test method II (50 rpm, 900ml of pH 6.8 buffer solution) show that the total drug release time is 8-24 hours, and n-value defined in the following equation is 0.8 to 1.2:
Y=ktⁿ (Y: amount of drug released (%), k: rate constant, n: release exponent, t: time).

10. The sustained-release tablet according to claim 9, wherein the said granules containing doxazosin mesylate include hydroxypropylmethyl cellulose having viscosity of 7,500 to 14,000 cps by 3 to 30 w/w % and hydroxypropylmethyl cellulose having viscosity of 40 to 60 cps by 15 to 30 w/w %.

11. The sustained-release tablet according to claim 9, wherein the granules containing doxazosin mesylate account for 40 to 80 w/w % in the total tablet weight.

12. The sustained-release tablet according to claim 9, wherein the post-mixed hydroxypropylmethyl cellulose having viscosity of 40 to 60 cps in b) is contained by 10 to 40 w/w % in total tablet weight.
